(19) United States
(12) Patent Application Publication
(10) Pub. No.: US 2007/0269380 A1
(43) Pub. Date: Nov. 22, 2007

(54) METHOTREXATE-MODIFIED NANOPARTICLES AND RELATED METHODS

(75) Inventors: Miqin Zhang, Bothell, WA (US); Shian-Jy Wang, Hsinchu (TW); Nathan Kohler, Providence, RI (US); Yuh-Jiuan Lin, Taipei County (TW); Conroy Sun, Kirkland, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignees: Washington, University of, Seattle, WA (US); Industrial Technology Research Institute, Hsinchu (TW)

(21) Appl. No.: 11/548,148

(22) Filed: Oct. 10, 2006

Related U.S. Application Data
(60) Provisional application No. 60/725,913, filed on Oct. 11, 2005.

(51) Int. Cl. A61K 31/4985; A61K 9/14; A61P 35/00; C07D 475/08; G01N 33/574

(52) U.S. Cl. 424/9.32; 424/489; 424/9.3; 435/325; 435/7.23; 514/249; 544/260; 977/773

(57) ABSTRACT

Methotrexate-modified nanoparticles that target tumors, compositions that include the nanoparticles, methods of imaging tissues using the nanoparticles, and methods for treating tissues using the nanoparticles.

### CROSS-REFERENCE TO RELATED APPLICATION

This application claims the benefit of U.S. Provisional Application No. 60/725,913, filed Oct. 11, 2005, incorporated herein by reference in its entirety.

### STATEMENT OF GOVERNMENT LICENSE RIGHTS

This invention was made with government support under Contract No. NO1-C037122 awarded by National Institute of Health. The Government has certain rights in the invention.

### BACKGROUND OF THE INVENTION

Over the past several years, there has been a growing interest in developing nanoparticle-based targeting agents for tumor diagnostics and therapeutics. It is recognized that with these targeting agents, tumors or other lesions can be detected at the cellular or molecular level. Two major applications associated with these systems are magnetic resonance imaging (MRI) and controlled drug release (CDR).

Magnetic resonance imaging (MRI) is an appealing non-invasive approach for early cancer diagnostics and therapeutics. While the imaging capabilities of these instruments have revolutionized imaging technology, the resolution of the instrument is limited to the elucidations of lesions within the body on the order of 1 mm with a clinical scanner. This limitation of the instrument has led to the development of several types of contrast enhancement agents including magnetite/dextran-based nanoparticles and chelated gadolinium contrast agents, which are currently available on the market and used widely in clinical applications. However, gadolinium (Gd) complex contrast agents are effective only when present in millimolar concentrations. Because of the superparamagnetic property, iron oxide nanoparticles have been found effective in nanomolar concentrations and can better serve as contrast enhancement agents for MRI.

Controlled drug release (CDR) has been the focus of many researchers in both academic and industrial settings for years. The science and technology utilizing liposomes as drug carriers have recorded major advances in the past decade. Interest in liposomes is directed upon their lipid-bilayer vesicular structure capable of encapsulating drugs and interacting with living cells. Nanoparticles as drug carriers are more attractive in view of their high tissue permeability, high colloidal stability, high carrier capacity, feasibility of incorporation of both hydrophilic and hydrophobic substances, feasibility of variable routes of administration, and small size. In CDR, nanoparticles function as drug carriers delivering and releasing drugs into target cells, offering the advantage of targeted or site-specific delivery of chemotherapeutics and other drugs to affected cells over an extended period, thereby increasing efficacy while reducing toxic side effects. Drugs can be grafted onto nanoparticles via physical adsorption, ionic bonding, and covalent bonding. Covalent bonding of drugs on nanoparticles is usually favored because bond strength makes nanoparticle-drug conjugates highly stable and therefore most likely to be disrupted only under harsh environment such as inside lysosomes.

The combination of MRI and CDR technologies by using nanoparticles may allow simultaneous diagnosis and treatment of the diseased tissues. Nanoparticle system in CDR needs to have an effective mechanism of drug release within the target cells. In addition, whether nanoparticles were serving as contrast agents or drug carriers, both applications rely on the efficiency of specific targeting by the nanoparticle systems.

However, identification of specific target agents and drugs which are capable of being released inside target cells remain as a challenge and is the central focus of the current studies in the field. Folic acid (FA) is generally recognized as an effective tumor targeting agent. Folate receptors are over-expressed on the cell membranes of many cancer cells including ovarian, endometrial, colorectal, breast, lung, renal cell carcinomas, brain metastases derived from epithelial cancers, and neuroendocrine carcinomas. Compared with widely-used antibodies which are bulky and difficult to cross the cell membrane, FA has short chains and a small size, and thus facilitates the internalization of nanoparticles. FA is stable, non-immunogenic, inexpensive and, in addition, has a very high affinity for its cell surface receptor.

Delivery of chemotherapeutic agents to target cells is not sufficient to induce cell death. Once the chemotherapeutic drug has been released inside the cell, it must be retained within the cell at concentrations sufficient to inhibit cell growth and function such as biosynthesis of expressed proteins. Methotrexate (MTX), an analogue of folic acid, exhibits not only a targeting role as folic acid, but also a therapeutic effect to many types of cancer cells that over express folate receptors on their surface.

Methotrexate is one of the most widely utilized drugs for the treatment of various forms of cancers. It has been utilized for the treatment of several forms of cancer for decades, including leukemias, breast cancer, head and neck cancer, lymphomas, and carcinomas. However, the clinical application of this drug is limited by its toxic dose-related side effects and drug resistance by target cells. The lack of selectivity of the low molecular weight methotrexate is closely related to its pharmacokinetic properties, i.e., short half-life in the bloodstream and rapid diffusion throughout the body resulting in an essentially uniform tissue distribution.

For the past decade, advances have been made in linking methotrexate drugs to a macromolecular carrier system to alter the pharmacokinetic behavior, enhance tumor targeting, reduce toxicity, and overcome drug resistance mechanisms. For example, when methotrexate was conjugated on human albumin, its circulation half-life in blood increased to 19 days, in contrast to the half-life of 2-3 hours of free methotrexate in humans. Poly(amido amine) dendrimers (PAMAM) have been synthesized as chemotherapeutic drug carriers for methotrexate. The conjugation of methotrexate combined with the targeting probe folic acid to the PAMAM nanoparticle facilitated enhanced cytotoxicity in mice bearing KB tumors. In addition, researchers have developed methotrexate conjugates consisting of polyglutamic acid or polyethylene glycol which retains a higher concentration of methotrexate within the cell. These conjugates have been shown to increase cellular cytotoxicity and increase cellular mortality. Despite the success of these conjugates in vitro, there is still no clinical method of detecting the levels of methotrexate taken up by the target cells, which may reduce the efficacy of the treatment.

Methotrexate has been conjugated to gelatin or polygluteraldehyde to improve the level of methotrexate taken up by the tumor cells. These methods of drug delivery can provide sustained release of methotrexate and improve the efficacy of treatment. However, the large size of the drug conjugate does not facilitate intravenous drug delivery. To be effective, the drug carrier must be at a size sufficient to perfuse out of the blood stream to reach the target cell of interest. Thus, the large size of these conjugates limits the administration of the drug carriers to direct injection into the tumor site.

While targeted drug delivery is critical to achieving effective therapy and reducing side effects, a strategy to monitor carrier distribution and tumor evolution under treatment may prove indispensable in both research and clinical settings. Magnetic resonance imaging (MRI) is recognized to be a non-invasive technique used to diagnose and monitor tumor growth in patients with cancer. Magnetic iron oxide nanoparticles have been FDA approved for MRI contrast enhancement for liver metastases in vivo and tested for contrast enhancement of many forms of cancer. Additionally, to be effective as a drug carrier, the issue of retention and continued function of the drug carrier in the target cells becomes critically important.

Despite the advances in the use of nanoparticles as contrast agents and drug carriers noted above, and despite the intensive research in the field to improve the efficacy of methotrexate, a need exists for nanoparticle-based methotrexate delivery systems that enable real-time monitoring of drug delivery to the target diseased tissue. The present invention seeks to fulfill this need and provides further related advantages.

### SUMMARY OF THE INVENTION

In one aspect, the invention provides a methotrexate-modified nanoparticle, comprising:

(a) a core having a surface, the core comprising a material having magnetic resonance imaging activity;

(b) a methotrexate; and

(c) a linker covalently coupling the methotrexate to the surface.

The core can be made of a material having magnetic resonance imaging activity comprises a metal oxide selected from the group consisting of ferrous oxide, ferric oxide, silicon oxide, polycrystalline silicon oxide, aluminum oxide, germanium oxide, zinc selenide, tin dioxide, titanium dioxide, indium tin oxide, gadolinium oxide, and mixtures thereof.

In one embodiment, the core comprises a material selected from the group consisting of silicon nitride, stainless steel, titanium, and nickel titanium, and mixtures thereof.

The linker can include an alkyl moiety or a poly(ethylene glycol) moiety.

Methotrexate is linked to the surface through a pH sensitive covalent bond.

In another aspect of the invention, compositions that include the particles of the invention are provided. In one embodiment, the composition includes a nanoparticle suitable for administration to a human or an animal subject. The composition can include an acceptable carrier.

In other aspects, the invention provides methods for using the nanoparticles.

In one embodiment, the invention provides a method for differentiating neoplastic tissue from non-neoplastic tissue, comprising:

(a) contacting a tissue of interest with a methotrexate-modified nanoparticle having affinity and specificity for tumor cells over-expressing folate receptor; and

(b) measuring the level of binding of the methotrexate-modified nanoparticle, wherein an elevated level of binding, relative to normal tissue, is indicative that the tissue is neoplastic.

In one embodiment, the invention provides a method for detecting tumor cells in a patient, comprising:

(a) contacting a tissue of interest with a methotrexate-modified nanoparticle having affinity and specificity for tumor cells over-expressing folate receptor; and

(b) measuring the level of binding of the methotrexate-modified nanoparticle, wherein an elevated level of binding, relative to normal tissue, is indicative that the tissue is neoplastic.

In one embodiment, the invention provides a method for detecting a tissue over-expressing folate receptor, comprising:

(a) contacting a tissue of interest with a methotrexate-modified nanoparticle having affinity and specificity for folate receptor; and

(b) measuring the level of binding of the methotrexate-modified nanoparticle, wherein an elevated level of binding, relative to normal tissue, is indicative of the presence of a tissue over-expressing folate receptor.

The invention provides methods for treating a tissue with the nanoparticles.

In one embodiment, the invention provides a method for treating cancer, comprising administering to a patient in need thereof an effective amount of a pharmaceutical composition comprising a methotrexate-modified nanoparticle and a pharmaceutically acceptable carrier.

In one embodiment, the invention provides a method for inhibiting invasive activity of neoplastic cells, comprising administering to neoplastic cells an effective amount of a pharmaceutical composition comprising a methotrexate-modified nanoparticle and a pharmaceutically acceptable carrier.

In one embodiment, the invention provides a method for treating a tissue over-expressing folate receptor, comprising administering to a patient in need thereof an effective amount of a pharmaceutical composition comprising a methotrexate-modified nanoparticle and a pharmaceutically acceptable carrier.

In one embodiment, the invention provides a method for treating a tumor in a patient, comprising:

(a) administering a pharmaceutical composition to a patient, wherein the pharmaceutical composition comprises a pharmaceutically acceptable carrier and an amount of a methotrexate-modified nanoparticle sufficient for treatment;

(b) monitoring the amount of methotrexate-modified nanoparticles delivered to the target tumor by magnetic resonance imaging; and

(c) accessing the efficacy of the treatment by analyzing the data from the magnetic resonance imaging.

In the above method, steps (a) and (b) or steps (a) to (c) may be repeated.

Methods for making the methotrexate modified nanoparticles are also provided.

In one embodiment, the invention provides a method for making a methotrexate-modified nanoparticle, comprising

(a) attaching a linker to a nanoparticle, wherein the nanoparticle comprises a core comprising a magnetic material; and

(b) attaching methotrexate to the linker to form the methotrexate-modified nanoparticle.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic description of the surface modification of magnetite nanoparticles with methotrexate;

FIG. 2 is a schematic description of the immobilization of PEG-methotrexate on magnetite nanoparticles;

FIG. 3 shows the TEM image and XRD pattern of superparamagnetic magnetite nanoparticles;

FIG. 4 shows FTIR spectra of (A) unmodified iron oxide nanoparticles, (B) APS-modified nanoparticles, (C) methotrexate-modified nanoparticles, and (D) free methotrexate;

FIG. 5 is the schematic representation of the intracellular uptake of methotrexate modified nanoparticles into breast cancer cells;

FIG. 6 shows the results of the release study of methotrexate from nanoparticles in simulated lysosomal pH conditions, as measured by UV absorbance;

FIG. 7 and 8 show the cellular viability, in terms of surviving fraction of MCF-7 and HeLa cells grown in the presence of methotrexate nanoparticles and soluble methotrexate over time;

FIG. 9 shows the preferential uptake of nanoparticle-methotrexate conjugate by breast cancer cells compared to cardiomyocyte;

FIG. 10 shows the TEM images of (A) MCF-7 control cells, (B) MCF-7 cells grown with methotrexate-nanoparticles, (C) HeLa control cells, and (D) HeLa cells grown with methotrexate-nanoparticles.

FIG. 11 is the FTIR spectroscopy of (A) native nanoparticles, (B) amine-terminal PEG nanoparticles, (C) methotrexate-coated nanoparticles, and (D) standard methotrexate;

FIG. 12 is the leucovorin (LV) rescue analysis of 9L cells grown in culture following exposure to 1 μg/mL of free methotrexate;

FIG. 13 shows the results from the leucovorin (LV) rescue control study of 9L cells exposed to 0.1 mg/mL NP-PEG-methotrexate and 0.22 μg/mL free methotrexate grown in culture;

FIG. 14 shows intracellular uptake of NP-PEG-methotrexate and NP-dextran nanoparticles in 9L cells following a 2 hour incubation time;

FIG. 15 shows the T₂-weighted spin-echo MR phantom images of 9L cells and sample holder schematic;

FIG. 16 shows T₂ relaxation analysis of 9L cell samples labeled with NP-PEG-methotrexate and NP-dextran conjugates; and

FIG. 17 shows the TEM images of 9L cells incubated with NP-PEG-methotrexate conjugates (A) for 24 hours and (B) 144 hours following Leucovorin rescue.

### DETAILED DESCRIPTION OF THE INVENTION

The present invention provides methotrexate-modified superparamagnetic nanoparticles capable of targeting tumors and enabling real-time monitoring of drug delivery to the target site, compositions that include the nanoparticles, methods of differentiating neoplastic tissues using the nanoparticles, methods of detecting tumor cells using the nanoparticles, methods for treating cells expressing folate receptor using the nanoparticles, methods for treating patients using the nanoparticles, and methods for making the nanoparticles.

In one aspect, the invention provides a methotrexate-modified nanoparticle comprising:

(a) a core having a surface, the core comprising a material having magnetic resonance imaging activity;

(b) a methotrexate; and

(c) a linker covalently coupling the methotrexate to the surface.

The particle includes a core having a surface that can be reacted with a linker. The particles can be core-shell particles in which the core is a material different from the shell. In one embodiment, the surface or shell comprises hydroxyl groups that are reactive toward the silane compounds.

The core includes a material having magnetic resonance imaging activity. Suitable materials having magnetic resonance imaging activity include metal oxides, such as ferrous oxide, ferric oxide, silicon oxide, polycrystalline silicone oxide, aluminum oxide, germanium oxide, zinc selenide, tin dioxide, titanium dioxide, indium tin oxide, and gadolinium oxide. Mixtures of one or more metal oxide can be used. In one embodiment, the core is a iron oxide nanoparticle.

In addition to magnetic materials, the core can include non-magnetic materials, such as silicon nitride, stainless steel, titanium, and nickel titanium. Mixtures of one or more non-magnetic materials can also be used.

The core of the particles useful in making the particles of the invention have a diameter of from about 5 nm to about 20 nm.

The particles of the invention can be nanoparticles having particle diameter of from about 50 nm to about 200 nm.

The particles of the invention include an amount of methotrexate sufficient to direct the nanoparticle to the desired site of action. The particles may include from about 20 to about 500 methotrexates/particle. In one embodiment, the particles include about 20-50 methotrexates/particle. In one embodiment, the particles include about 420 methotrexates/particle.

In the invention, methotrexate is attached to the particle surface through a bifunctional linker. The linker includes a straight or branched chain, including alkyl or poly(ethylene glycol) (PEG) moieties, such as 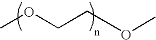
and n is an integer from about 10 to about 1000. In one embodiment, the linker is 3-aminopropyl trimethoxysilane (APS). In one embodiment, the linker is a trifluoroethylester (TFEE) terminated silane including a 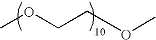
moiety.

The core or shell surface can be covered with functional groups, including hydroxyl group, amino group, tosylate, halide and carboxylic groups. In one embodiment, the surface is covered with hydroxyl groups. In one embodiment, the surface is covered with amino groups.

Methotrexate can be attached to the surface through a pH sensitive covalent bond with the linker. Suitable pH sensitive covalent bonds include bonds that cleave at a pH of from about 2 to about 5. Representative pH sensitive bonds include peptide bonds, ester bonds, hydrazide bonds, and aromatic azo bond, particularly amide or ester bonds.

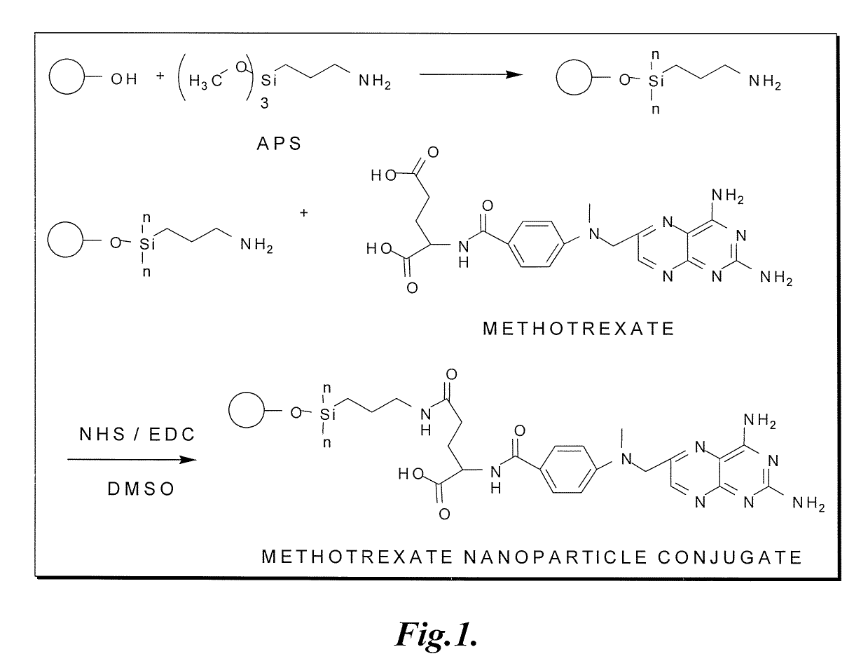

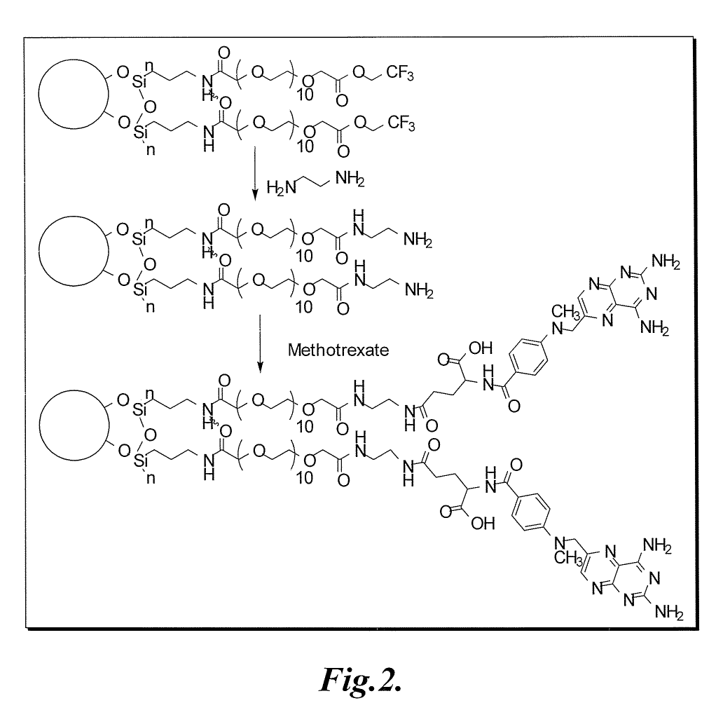

Methotrexate can be attached to the surface through a peptide bond. In one embodiment, the iron oxide nanoparticles were first surface-modified with 3-aminopropyltrimethoxysilane (APS) to form a self-assembled monolayer (SAM) and subsequently conjugated with methotrexate through a peptide bond formed between the carboxylic acid end groups on methotrexate and the amine groups on the particle surface (FIG. 1). The methotrexate conjugation reaction may occur through either the alpha or beta carboxylic acid groups on the glutamic acid residue. In one embodiment, methotrexate was immobilized on the nanoparticle surface via a poly(ethylene glycol) self-assembled monolayer (PEG SAM) as shown in FIG. 2. The bifunctional PEG silane was covalently bound on Fe₃O₄ nanoparticles with hydroxyl groups via a silane terminus, leaving trifluoroethylester-terminal to conjugate the amine groups of methotrexate through an amide bond. The trifluoroethylester-terminal group allows covalent functionalization of methotrexate to the nanoparticle facilitating targeted cellular uptake of the nanoparticle.

In another aspect, the invention provides a method for making a methotrexate-modified nanoparticles. In one embodiment, the methotrexate-modified nanoparticles are made by,

(a) attaching a linker to a nanoparticle, wherein the nanoparticle comprises a core comprising a magnetic material; and

(b) attaching methotrexate to the linker to form the methotrexate-modified nanoparticle.

The nanoparticle was synthesized by reacting a nanoparticle core with bifunctional linkers followed by functionalizing the linkers with methotrexate.

In one embodiment, methotrexate was attached to the surface of iron oxide nanoparticles through a 3-aminopropyltrimethoxy silane (APS) linker to form NP-propyl-methotrexate nanoparticles. As used herein, “NP” refers to nanoparticle. Magnetite, Fe₃O₄, nanoparticles were surface-modified with methotrexate via a chemical scheme outlined in FIG. 1. The nanoparticles were first surface-modified with 3-aminopropyltrimethoxysilane (APS) to form a self-assembled monolayer (SAM) and subsequently conjugated with methotrexate through amidation between the carboxylic acid end groups on methotrexate and the amine groups on the particle surface. The methotrexate conjugation reaction may occur through either the alpha or beta carboxylic acid groups on the glutamic acid residue.

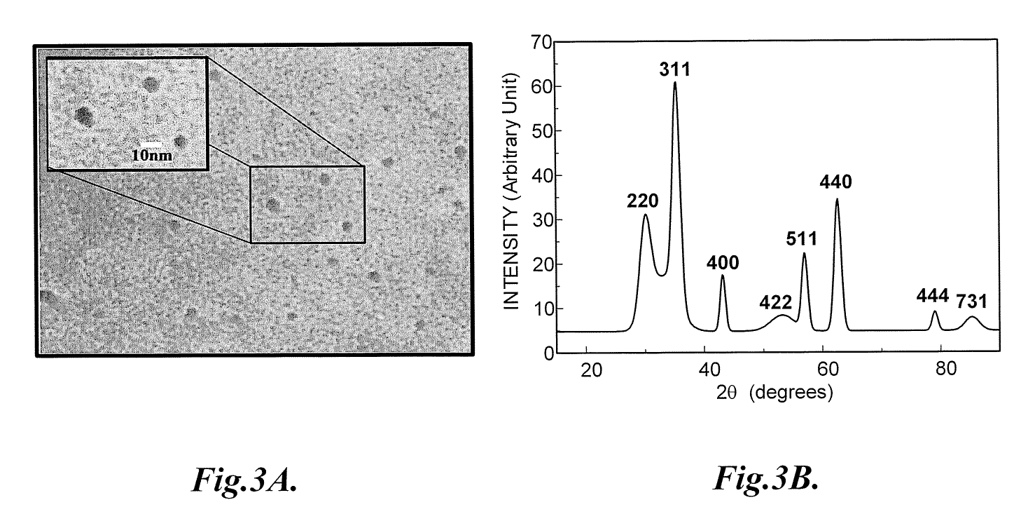

The TEM image in FIG. 3A shows that the particles as synthesized are well dispersed and have a uniform shape and size distribution. The X-Ray powder diffraction pattern shown in FIG. 3B for the nanoparticles agrees with the pattern of magnetite nanoparticles listed in ASTM XRD standard card (19-0629). The nanoparticle size evaluated from the diffraction pattern using the Scherrer formula is about 10 nm, consistent with the TEM estimation shown in FIG. 3A.

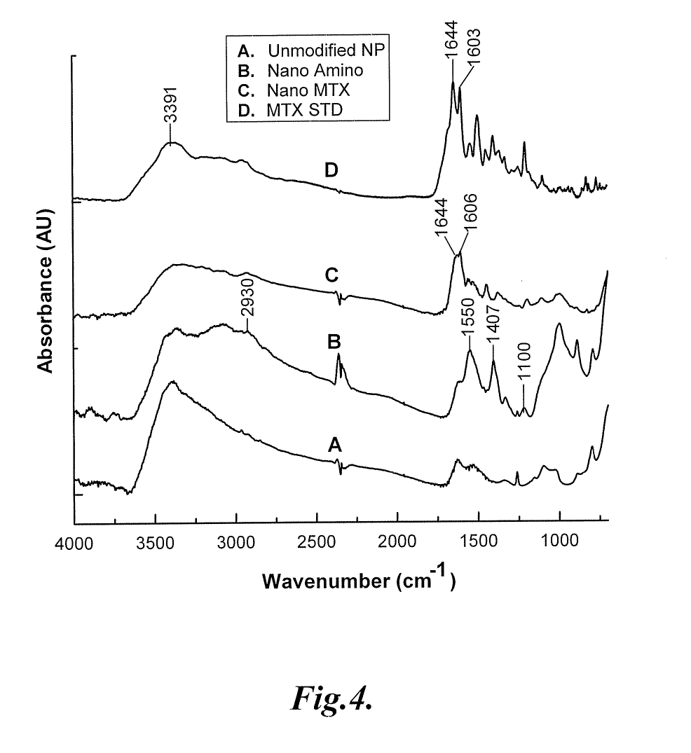

FTIR spectroscopy was used to confirm that methotrexate was successfully immobilized on the nanoparticles. FTIR spectra of unmodified and methotrexate modified iron oxide nanoparticles are shown in FIG. 4. The unmodified superparamagnetic nanoparticles show a broad band at 3300 cm⁻¹ indicative of the presence of —OH groups on the nanoparticle surface. For nanoparticles modified with 3-aminopropyltrimethoxysilane, the peaks at 1550 cm⁻¹ and 1407 cm⁻¹ indicate the presence of the primary amine on the nanoparticle surface. The peak at 1100 cm⁻¹ indicates Si—O bonding on the nanoparticle surface. Further, the peak at 2930 cm⁻¹ indicates the —CH stretch present in the APS. Standard methotrexate shows the characteristic IR absorption peaks of 1644 cm⁻¹ and 1603 cm⁻¹. Spectra of nanoparticles modified with methotrexate following APS immobilization show increased absorbance at 1606 cm⁻¹ and appearance of a new peak at 1550 cm⁻¹ indicative of the presence of methotrexate on the nanoparticle through amide bonds within the methotrexate structure and amide bonding between APS and methotrexate.

In one embodiment, methotrexate was attached to the surface of iron oxide nanoparticles through a trifluoroethylester (TFEE)-terminal PEG linker to form NP-PEG-methotrexate nanoparticles. The chemical scheme for conjugating methotrexate onto the surface of the nanoparticle through an amide bond is shown in FIG. 2. This method of methotrexate immobilization by covalent binding creates a linker between the PEG SAM and the glutamic acid residue of the methotrexate molecule, which is stable under intravenous conditions. Following the immobilization of the trifluoroethylester (TFEE)-terminal PEG SAM on the nanoparticle surface, the TFEE terminus of the PEG SAM is converted to a primary amine through ethylene diamine. The methotrexate is then covalently immobilized to the PEG chain terminus through a succinimidyl ester reaction.

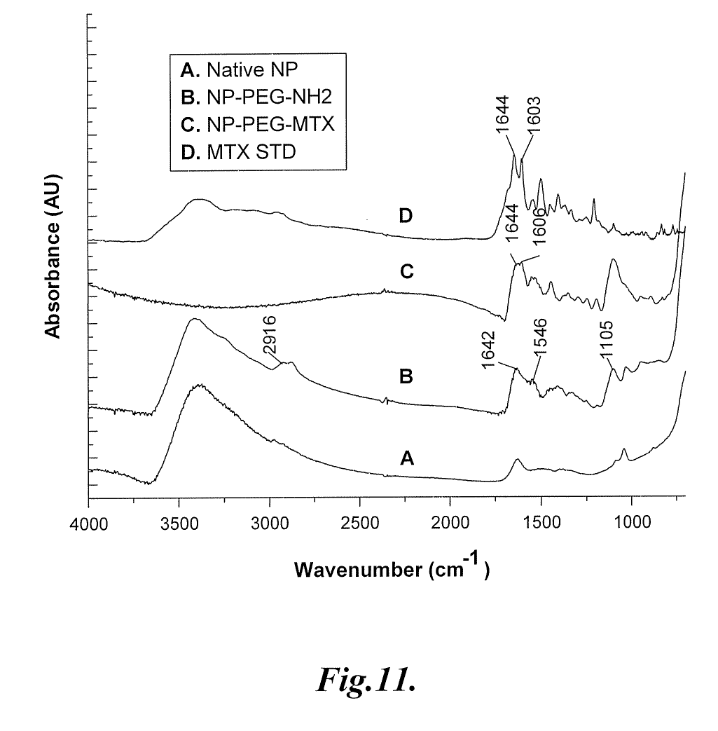

Successful immobilization of the PEG amine and methotrexate on the nanoparticle was confirmed by FTIR analysis as shown in FIG. 11. Following the immobilization of the amine-terminal PEG silane (B), an intensity increase was seen in the -CH stretch at 2916 cm⁻¹ and the amide carbonyl groups at 1642 and 1546 cm⁻¹. Additionally, the presence of the —SiO peak at 1105 cm⁻¹ indicates the covalent immobilization of the PEG silane on the particle surface. Following the immobilization of methotrexate on the nanoparticle (C), peaks at 1644 and 1606 cm⁻¹ confirmed the presence of the methotrexate on the particle surface. For comparison, a spectrum for free methotrexate (D) is also shown in the figure.

In another aspect of the invention, compositions that include the particles of the invention are provide. In one embodiment, the composition include a nanoparticle suitable for administration to a human or an animal subject. The composition can include an acceptable carrier. In one embodiment, the composition is a pharmaceutically acceptable composition and includes a pharmaceutically acceptable carrier. As used herein the term “carrier” refers to a diluents (e.g., saline, PBS) to facilitate the delivery of the particles.

The compositions in the invention include an amount of methotrexate-modified nanoparticles sufficient for treatment. The amount of nanoparticles in the compositions, measured by the iron concentration, can be from about 200 to about 2000 μg Fe/mL. In one embodiment, the amount of methotrexate-modified nanoparticles in the composition is about 50 μg Fe/mL. In one embodiment, the amount of methotrexate-modified nanoparticles in the composition is about 100 μg Fe/mL.

In other aspects, the invention provides methods for using the nanoparticles.

In one embodiment, the invention provides a method for differentiating neoplastic tissue from non-neoplastic tissue. In the method, neoplastic tissue is differentiated from non-neoplastic tissue by:

(a) contacting a tissue of interest with a methotrexate-modified nanoparticle having affinity and specificity for tumor cells over-expressing folate receptor; and

(b) measuring the level of binding of the methotrexate-modified nanoparticle, wherein an elevated level of binding, relative to normal tissue, is indicative that the tissue is neoplastic.

In one embodiment, the invention provides a method for detecting tumor cells in a patient. In the method, the tumor cells are detected by:

(a) contacting a tissue of interest with a methotrexate-modified nanoparticle having affinity and specificity for tumor cells over-expressing folate receptor; and

(b) measuring the level of binding of the methotrexate-modified nanoparticle, wherein an elevated level of binding, relative to normal tissue, is indicative that the tissue is neoplastic.

In one embodiment, the invention provides a method for detecting a tissue over-expressing folate receptor. In the method, the tissue over-expressing folate receptor is detected by:

(a) contacting a tissue of interest with a methotrexate-modified nanoparticle having affinity and specificity for folate receptor; and

(b) measuring the level of binding of the methotrexate-modified nanoparticle, wherein an elevated level of binding, relative to normal tissue, is indicative of the presence of a tissue over expressing folate receptor.

In the methods above, measuring the level of binding of the methotrexate-modified nanoparticle comprises magnetic resonance imaging.

The invention provides method for treating cancer using the nanoparticles.

In one embodiment, the invention provides a method for treating a tumor in a patient, comprising administering to a patient in need thereof an effective amount of a pharmaceutical composition comprising a methotrexate-modified nanoparticle and a pharmaceutically acceptable carrier.

In one embodiment, the invention provides a method for treating a tumor, comprising administering to a patient in need thereof an effective amount of a pharmaceutical composition comprising a methotrexate-modified nanoparticle and a pharmaceutically acceptable carrier.

The methotrexate-modified nanoparticles of the invention can be used to treat various cancers including ovarian, endometrial, colorectal, breast, lung, renal cell carcinomas, brain metastases derived from epithelial cancers, or neuroendocrine carcinomas.

In one embodiment, the invention provides a method for inhibiting invasive activity of neoplastic cells, comprising administering to neoplastic cells an effective amount of a pharmaceutical composition comprising a methotrexate-modified nanoparticle and a pharmaceutically acceptable carrier.

In one embodiment, the invention provides a method for treating a tissue over-expressing folate receptor, comprising administering to a patient in need thereof an effective amount of a pharmaceutical composition comprising a methotrexate-modified nanoparticle and a pharmaceutically acceptable carrier.

In one embodiment, the invention provides a method for treating a tumor in a patient. The method includes the steps of,

(a) administering a pharmaceutical composition to a patient, wherein the pharmaceutical composition comprises a pharmaceutically acceptable carrier and an amount of a methotrexate-modified nanoparticle sufficient for treatment;

(b) monitoring the amount of methotrexate-modified nanoparticles delivered to the target tumor by magnetic resonance imaging; and

(c) accessing the efficacy of the treatment by analyzing the data from the magnetic resonance imaging.

In one embodiment, the method of treatment further comprises administering a pharmaceutical composition to a patient, wherein the pharmaceutical composition comprises a pharmaceutically acceptable carrier and an amount of a methotrexate-modified nanoparticle sufficient for treatment; monitoring the amount of methotrexate-modified nanoparticles delivered to the target tumor by magnetic resonance imaging; and accessing the efficacy of the treatment by analyzing the data from the magnetic resonance imaging. By using magnetic resonance imaging to monitor the amount of nanoparticles at the target tumor site, physician could gain the knowledge of the drug concentration inside the tumor cells as well as the physiological changes at the tumor site, access the efficacy of the treatment, and make proper and prompt adjustment to the drug dosage regime if needed. In one embodiment of the method, the steps of monitoring and assessing can be repeated. In another embodiment, the steps of administering, monitoring, and assessing can be repeated.

The methods of the invention are applicable to human and animal subjects.

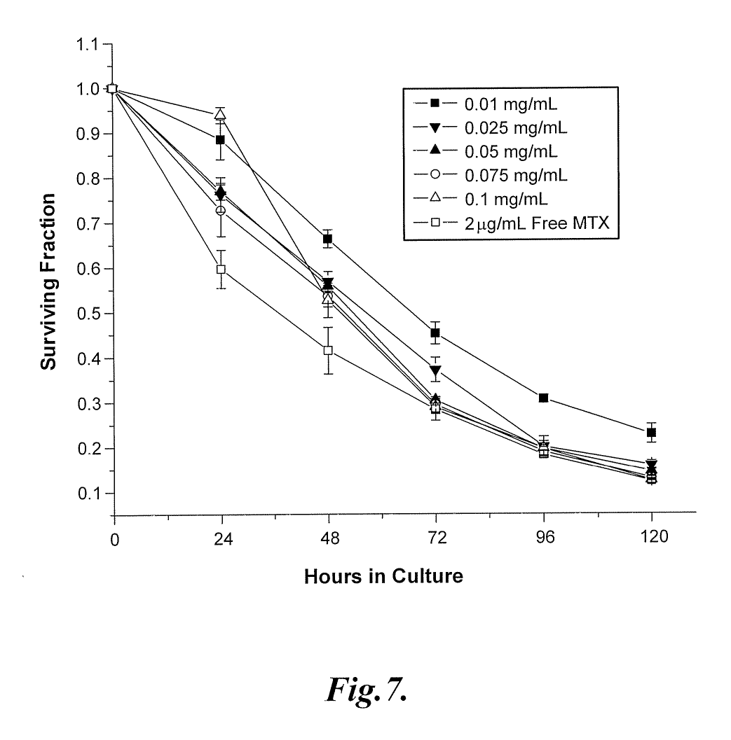

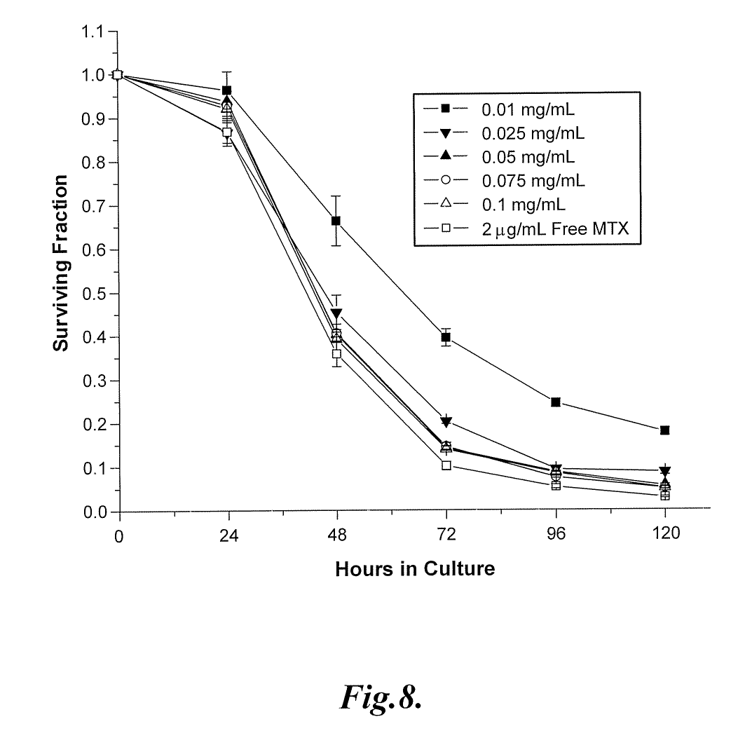

Once the release of methotrexate from the nanoparticle surface had been verified, the effectiveness of superparamagnetic nanoparticles to serve as drug carriers was evaluated in vitro. Human breast cancer cells (MCF-7) and human cervical cancer cells (HeLa) were grown in the presence of free methotrexate and NP-propyl-methotrexate nanoparticles. FIG. 7 and 8 show the cellular viability, in terms of surviving fraction of MCF-7 and HeLa cells grown in the presence of methotrexate nanoparticles and soluble methotrexate over time. After 120 hours in culture, all cells demonstrated a notable downward trend in viability indicative of the cytotoxicity of both the methotrexate nanoparticles and soluble methotrexate over time. In both the MCF-7 cells and HeLa cells, the methotrexate-modified nanoparticles and the soluble methotrexate show a similar reduction in cell viability establishing the ability of the cells to cleave methotrexate in the lysosome, thus allowing the freed methotrexate to reduce cellular viability. In addition, a dose response for the methotrexate-modified nanoparticles was elucidated through the viability results. For the MCF-7 cells, concentrations of methotrexate modified nanoparticles at or above 0.025 mg/mL showed a similar reduction in cellular viability. HeLa cells demonstrated a similar dose response, namely, for concentrations of methotrexate-modified nanoparticles at or higher than 0.05 mg/mL, the cells showed a statistically equivalent reduction in cellular viability. It should be noted that for the HeLa cells, 2 μg/mL of soluble methotrexate demonstrated a slightly higher cytotoxicity than the methotrexate-modified nanoparticles.

Methotrexate is analog of folic acid, which contains an amino group at position 4 in the ring of pteridine ring leading to a critical change in the structure of folic acid allowing for tight binding of dihydrofolate reductase (DHFR), a critical enzyme in the folic acid cycle, key to regulating homeostasis. When delivered in high enough doses, methotrexate causes the toxic buildup of cellular intermediates, reducing cellular viability and ultimately causing cellular mortality.

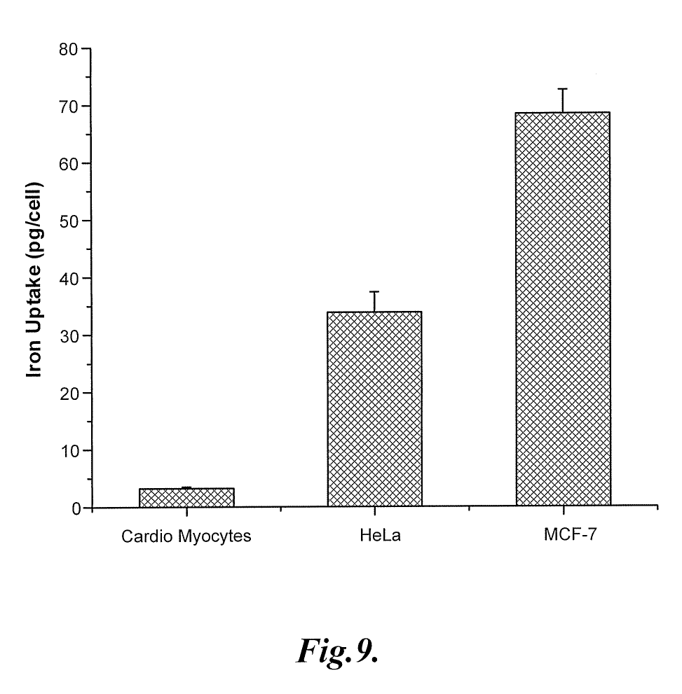

Cellular uptake by target cells measures the specificity of a nanoparticle-conjugate for the target cells. Inductive coupled plasma (ICP) resonance spectroscopy was utilized to quantify the cellular uptake of nanoparticle conjugates into MCF-7, BT-20, and HT1080 cells in terms of iron concentration. The results shown in FIG. 9 demonstrate the specificity of the NP-propyl-methotrexate nanoparticles, for the human folate receptor and reduced folate carrier. To demonstrate the specify for cancer cells, rat primary cardiomyocyte (heart muscle) cells were used as a negative control. Following 2 hours in culture, the HeLa cells demonstrated an uptake approximately 10 times higher that the negative control (FIG. 9), while the MCF-7 cells demonstrated an uptake approximately 20 times higher that the primary cardiomyocyte cells in culture. This might be due to the high metabolic activity of HeLa and MCF-7 cells leading to the over expression of the folate receptor on the cell surface and the low metabolic activity of cardiomyocyte cells. Both MCF-7 and HeLa cells have also been shown to be positive for the reduced folate carrier by previous studies. The uptake of methotrexate is known to have at least two different carrier systems which include (1) the rescued folate carrier for which methotrexate and reduced folates have a higher affinity than folic acid and (2) the folate receptor for which folic acid has a higher affinity than that of methotrexate. Folic acid and methotrexate themselves are low molecular weight targeting molecules which have little ability to pass through the cellular membrane nonspecifically.

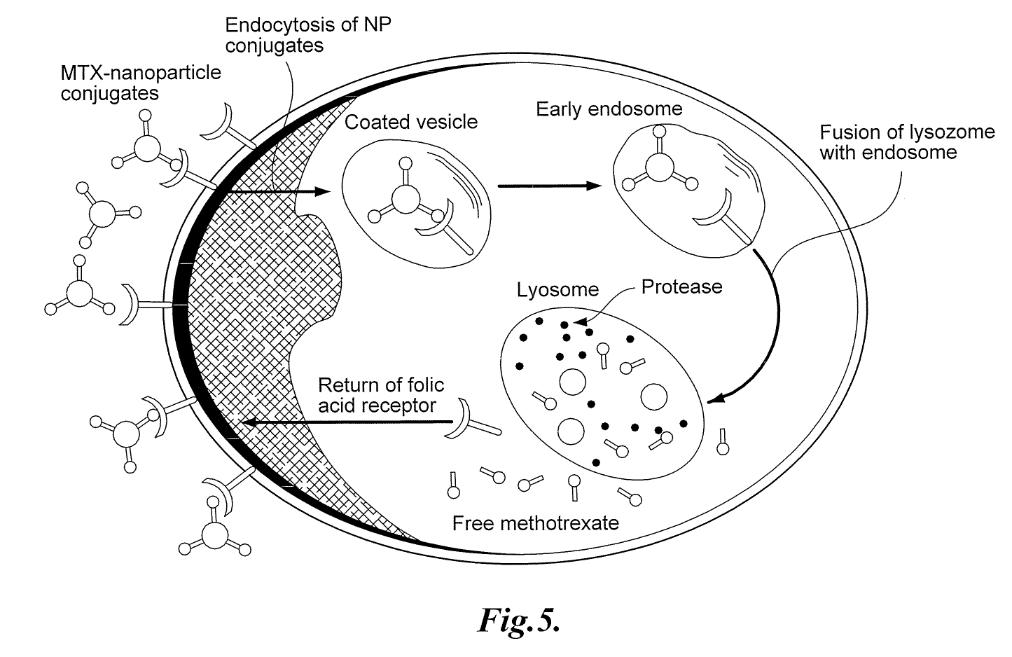

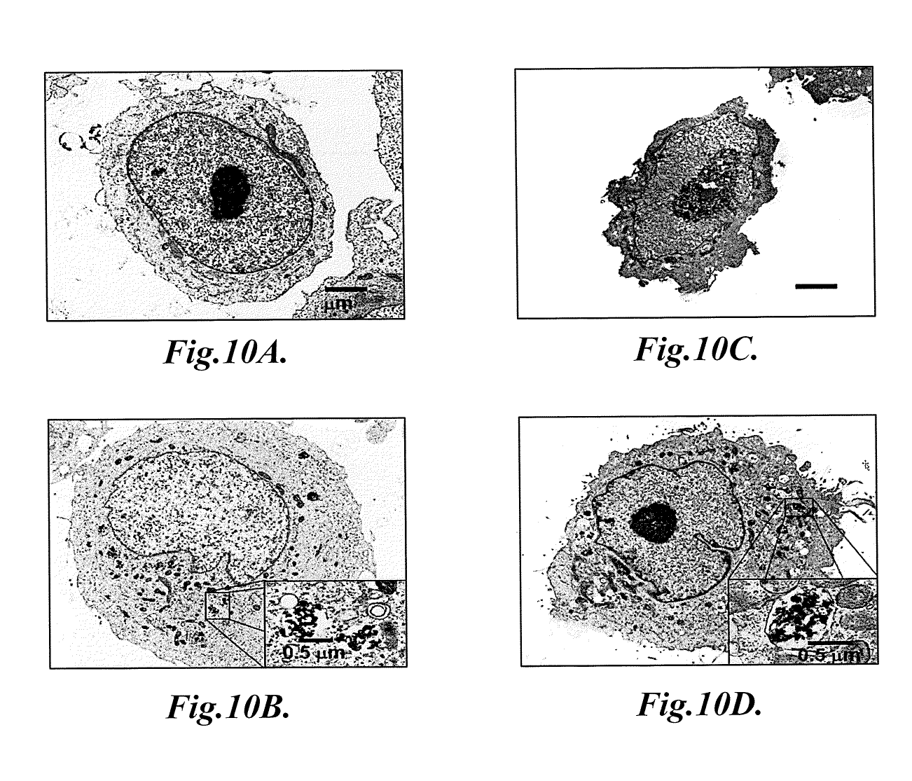

To further confirm that the methotrexate-nanoparticle conjugates were indeed internalized by the target cells rather than simply bound to the surface of the cells, and to visualize the location of the nanoparticles inside the cells after the internalization, TEM images were taken both on MCF-7 and HeLa cells that were cultured with NP-propyl-methotrexate nanoparticles and, for comparison, on their corresponding cells that were cultured without methotrexate-modified nanoparticles. FIG. 10 shows the images of methotrexate-nanoparticle treated MCF-7(B) and HeLa (D) cells, and comparative untreated MCF-7 (A) and HeLa (C) cells. This comparison provides evidence that a large number of methotrexate-nanoparticle conjugates accumulated in both MCF-7 and HeLa cells treated with methotrexate-nanoparticle conjugates and appeared as black dots scattered in the cell cytoplasm but not in the nuclei. A closer look at the images reveals that the majority of the internalized methotrexate-nanoparticle conjugates resided in the lysosomes of the cells (insets of B and D of FIG. 10), which supports the intracellular trafficking model illustrated in FIG. 5.

The cytotoxicity of the methotrexate modified PEG-nanoparticles (NP-PEG-methotrexate) to target cells was assessed in 9L glioma cells. To assess the biocompatibility of the NP-PEG-methotrexate conjugate and verify that methotrexate on NP-PEG-methotrexate conjugate was the true source of the cytotoxicity to target cells, Leucovorin rescue experiments were conducted, in which the cells cultured with NP-PEG-methotrexate were incubated with Leucovorin. Contrast enhancement of the NP-PEG-methotrexate conjugates for MRI detection was demonstrated in 9L cells by MRphantom imaging and relaxivity measurements, and a comparison was made with dextran stabilized nanoparticles (NP-dextran) serving as a control. Transmission electron microscopy was used to visualize the intracellular uptake and retention of the NP-PEG-methotrexate conjugates in target cells, and electron diffraction was used to examine the integrity of the magnetite crystal structure of NP-PEG-methotrexate conjugates in the cell following the intracellular uptake in a time course.

As noted above, the present invention provides a methotrexate-modified nanoparticle capable of targeting tumor cells over expressing folate receptor and detectable by magnetic resonance imaging (MRI).

The methotrexate-modified nanoparticles of the invention can release free methotrexate molecules once inside the target cells because of the pH sensitive bond linking methotrexate and the magnetic core. Although not wanting to be limited by the theory, it is believed that the mechanism of releasing methotrexate from the nanoparticle conjugate inside target cells is similar to folic acid. FIG. 5 conceptually illustrates the intracellular trafficking model of the uptake of methotrexate-modified nanoparticles into target cells. Following their uptake via receptor-mediated endocytosis, nanoparticles are transported to early endosomes. The endosomes then fuse with low pH lysosomes containing proteases which during normal cellular metabolism are responsible for the breakdown of proteins and other exogenous materials brought into the cell. These proteases then cleave the peptide bond between the methotrexate and the nanoparticle, allowing the methotrexate to be released from the particle surface inside the target cell. Once the methotrexate is free from the nanoparticle surface, it may enter the cellular cytosol. It is then assumed that methotrexate will be free to inhibit dihydrofolate reductase and stop the folic acid cycle reducing cellular viability.

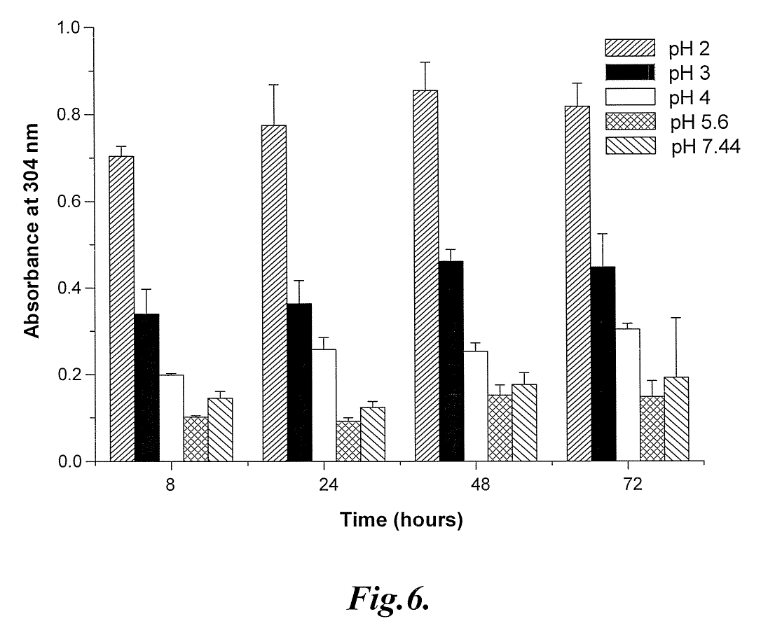

The number of methotrexate molecules immobilized on each nanoparticle was quantified using UV-vis absorbance data and the particle concentration data determined by ICP. From this analysis, the average number of methotrexate molecules per particle for nanoparticles with a 10 nm diameter was determined to be about 419. The release of methotrexate from the nanoparticle conjugate in simulated lysosomal condition (i.e., acidic pH and in the presence of proteases) was studied by UV spectroscopy. The amide bonds formed during the surface modification with methotrexate are between he glutamic acid residues of the methotrexate molecule and the amino terminal SAM. It was theorized that the proteases found in the lysosomal compartment may be capable of dehydrolyzing the peptide bond releasing free methotrexate into the cellular cytoplasm. To test this theory, the nanoparticles were incubated with crude protease solution at alternate pH conditions to facilitate hydrolysis, similar to conditions found in the lysosome. Results of methotrexate release from the nanoparticle surface under lysosomal conditions as measured by UV absorbance at 304 nm are shown in FIG. 6. Using the standard protease solution in buffer as a blank, it is seen that UV absorption increases with decreasing pH. The greatest UV absorption occurs at pH 2 which is most likely due to the greatest concentration of active protease at this pH. Studies were conducted from 12 to 72 hours to gain an understanding of the kinetics of the methotrexate release from the nanoparticles. However, the data suggest that methotrexate release occurred prior to the 12 hour interval, indicating that the protease readily cleaves the peptide bond. This study also indicates that there is some release of methotrexate from the nanoparticle at pH 4-7.44, which may be due to the presence of a small amount of active protease capable of cleaving the amid bond of the nanoparticle.

A methotrexate release study using UV spectroscopy was performed to confirm the successful cleavage of methotrexate from the NP-PEG-methotrexate nanoparticle surface and quantify the amount of methotrexate released from the nanoparticle surface. The methotrexate was cleaved from the PEG chain terminus by using proteinase K enzyme, a protease which cleaves amide bonds, and measured at 304 nm. By comparing the UV absorbance of methotrexate released from the nanoparticle to a standard linear fit curve of free methotrexate, a drug-release payload of 0.22 μg methotrexate per 0.1 mg of nanoparticles was obtained. A similar quantification strategy was reported using crude protease from bovine pancreas to cleave the amide bond between methotrexate and polymer drug carriers to quantify drug release.

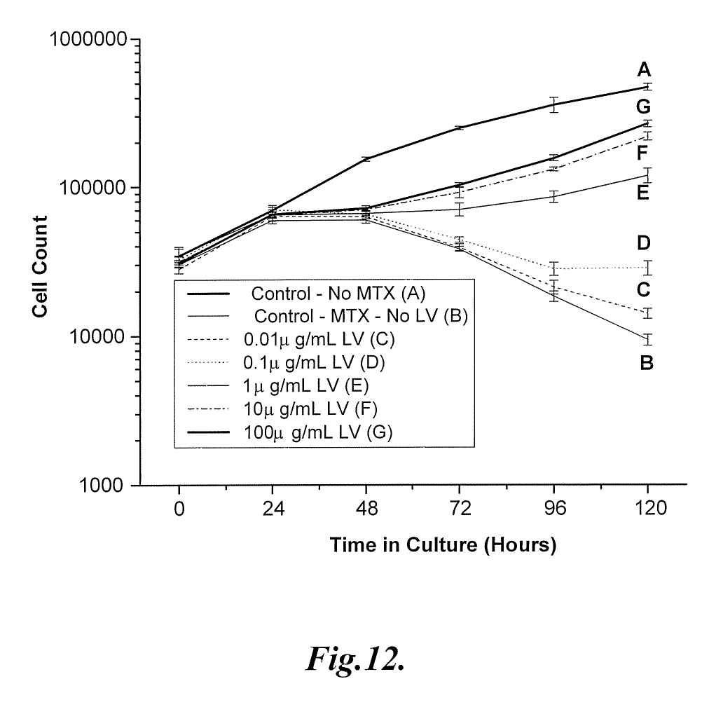

The mechanism with which NP-PEG-methotrexate conjugates induce apoptosis and biocompatibility of the nanoparticle conjugate were studied through leucovorin-induced rescue analysis, in which cells that had been exposed to NP-PEG-methotrexate were rescued by a leucovorin that have been proved to be effective to rescue many types of cells under induced cellular apoptosis. Methotrexate induces apoptosis in cells through inhibition of the folic acid cycle by acting on the enzyme, dihydrofolate reductase (DHFR). The substitution of a hydroxyl group for an amino group at position four of the pteridine ring transforms the molecule from a substrate of DHFR to a tight binder. By creating a tight binding complex, the folic acid cycle is inhibited leading to a buildup of folates in the inactive dihydrofolate form, depleting the intracellular pools of reduced folates. Leucovorin (folinic acid) is commonly used in high dose chemotherapy to rescue the folic acid cycle in healthy cells and reduce the deleterious side effect of the therapy. Leucovorin rescues the cell cycle by being actively metabolized into reduced folates, increasing the intracellular concentration of the reduced folates within the cytoplasm. In this study, to identify methotrexate on the NP-PEG-methotrexate conjugate being the source of cytotoxicity to target cells, Leucovorin was used as an antidote to NP-PEG-methotrexate cytotoxicity in the 9L cells. Previous studies used leucovorin to rescue MCF-7 and HeLa cells following exposure to free methotrexate, but no studies have been reported in using leucovorin for the 9L cell line. Thus, the first step in this study was to determine whether the 9L cells would respond to leucovorin induced rescue following the cellular exposure to free methotrexate. Based on the preliminary data, the 9L cells were exposed to methotrexate at a concentration of 1.0 mg/mL, a dosage sufficient to induce the apoptosis of 9L cells. Following exposure to methotrexate for 2 hours, the cells were counted and washed twice with PBS, and then cultured for up to 120 hours in culture media containing leucovorin at concentrations ranging from 0.01 μg/mL to 100 μg/mL to assess the level of leucovorin sufficient to rescue the cells and re-establish cell proliferation. The results are shown in FIG. 12. After incubation with leucovorin (LV) for 48 hours, only cells incubated with LV at a concentration of 1 μg/mL or higher showed continued proliferation. This indicates that LV is effective in rescuing 9L cells from methotrexate induced apoptosis, and that the sufficient concentration of LV to ensure the cell rescue for 9L cells is ˜10 μg/mL.

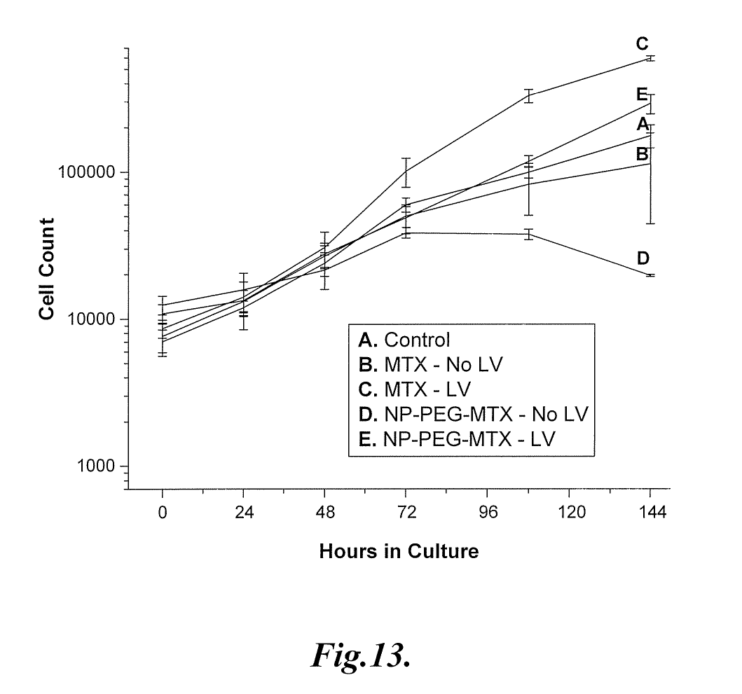

Once the ability of leucovorin to rescue 9L cells from free methotrexate was confirmed, 9L cells were incubated with NP-PEG-methotrexate at an iron concentration of 0.1 mg/mL as confirmed by ICP, which is equivalent to 0.22 μg/mL free methotrexate by UV analysis. In addition, free methotrexate at a concentration of 0.22 μg/mL was used as a control. Cells were first exposed to free methotrexate at a concentration of 0.22 μg/mL or NP-PEG-methotrexate conjugates for 24 hours and followed by leucovorin at various concentrations. The results of the leucovorin rescue of 9L cells exposed to NP-PEG-methotrexate and free methotrexate are shown in FIG. 13. Cells grown in the absence of methotrexate and leucovorin were used as a control. Cells cultured in the presence of NP-PEG-methotrexate but without leucovorin exhibited a marked reduction in viability (D). Cells cultured with both NP-PEG-methotrexate and leucovorin at a concentration of 10 μg/mL proliferated well over time (E) and exhibited a higher viability than the cells cultured with free methotrexate but without leucovorin (B). The difference in leucovorin induced rescue between the cells treated with the NP-PEG-methotrexate conjugate and with free methotrexate may be due to the enhanced intracellular concentrations of methotrexate in the cytosol following uptake of the NP-PEG-methotrexate conjugates. Cellular resistance to free methotrexate has been reported to be due to cellular efflux pumps. Conjugating methotrexate with high molecular weight PEG have been shown to decrease the cellular efflux of methotrexate, enhance the intracellular retention, and increase the cytoplasmic concentration of methotrexate. Free methotrexate has previously been conjugated to free PEG to enhance the circulation time and increase the cellular cytotoxicity. In this case, the drug was conjugated to a PEG SAM and nanoparticle, and thus, both of which may contribute to reduce the efflux of methotrexate from the cellular cytoplasm and increase the cytotoxicity of the conjugate.

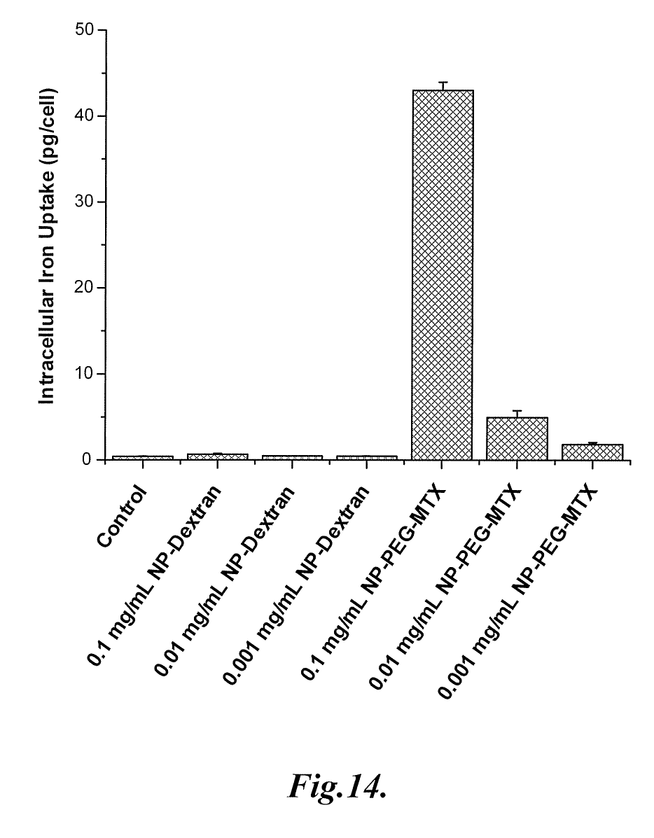

The intracellular uptake of NP-PEG-methotrexate conjugates in 9L glioma cells was quantified by ICP. Dextran-stabilized iron oxide nanoparticles (NP-dextran), a non-targeted MRI contrast agent, was used as a control. The results of the intracellular uptake experiments are shown in FIG. 14. The NP-PEG-methotrexate conjugates exhibited a concentration-dependent uptake by 9L cells. Cells incubated with NP-PEG-methotrexate conjugates at a concentration of 0.1 mg/mL demonstrated an 8-9 fold higher uptake than cells incubated with NP-PEG-methotrexate at a concentration of 0.01 mg/mL. Compared to the cells incubated with NP-PEG-methotrexate conjugate, cells incubated with dextran-coated nanoparticles took up little nanoparticles, and the uptake appeared independent of particle concentration as expected due to non-targeting nature of dextran-coated nanoparticles. It is expected that the NP-PEG-methotrexate conjugates are specific for the folate receptor and the reduced folate carrier allowing receptor mediated endocytosis of the conjugate into the cell. This specific internalization of the NP-PEG-methotrexate conjugate considerably increased the uptake of the nanoparticle as compared to the NP-Dextran conjugate.

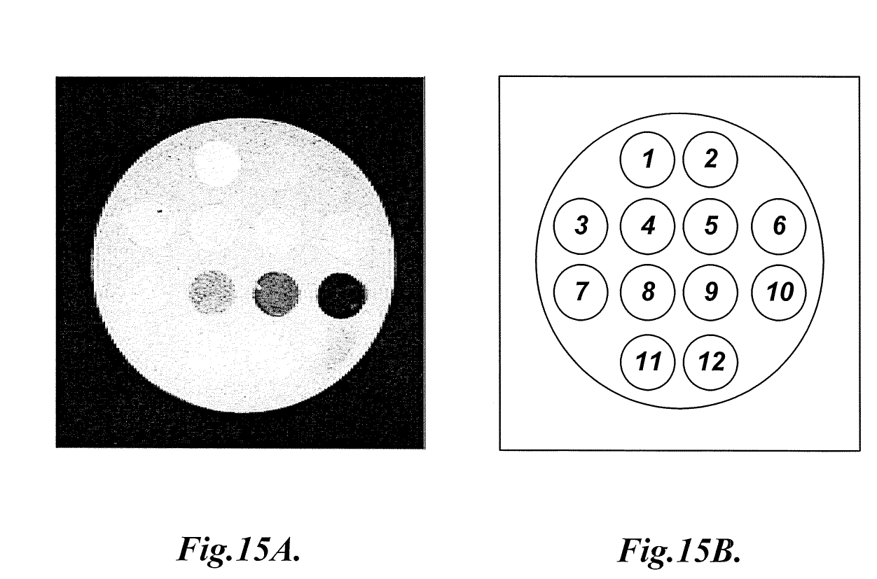

Magnetic resonance phantom imaging was used to assess the magnetic properties (thus, the detectability by MRI) of NP-PEG-methotrexate conjugates and to further confirm the intracellular nanoparticle uptake by target cells in addition to the ICP quantification shown above. MR phantom samples were prepared by suspending 9L cells incubated with NP-PEG-methotrexate or NP-dextran conjugates for two hours in agarose. T₂ weighted MR images of NP-PEG-methotrexate and NP-dextran are shown in FIG. 15. The NP-dextran conjugates show little change in MR contrast with increased particle concentration. In contrast, MR phantom images of the cells incubated with NP-PEG-methotrexate conjugates exhibited significant negative contrast enhancement as the concentration of the nanoparticle conjugate increased from 0.001 mg/mL to 0.1 mg/mL. This trend correlates well with the results of iron uptake into the 9L cells quantified by ICP (FIG. 14). The receptor mediated endocytosis of the NP-PEG-methotrexate conjugate increases the internalization of the conjugate and thus, the MRI contrast.

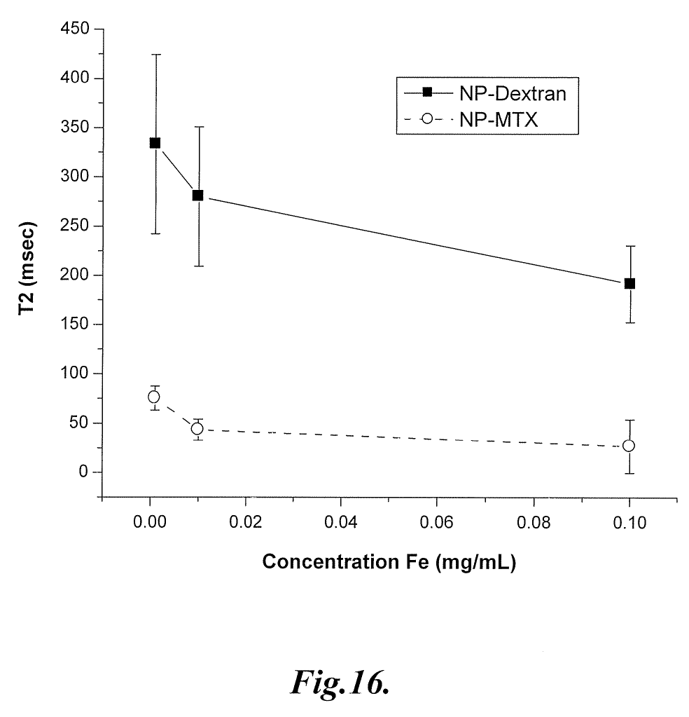

The relative relaxation times of the NP-PEG-methotrexate and NP-Dextran conjugates were quantified through T₂-weighted spin-echo MR images. FIG. 16 shows the T₂ relaxation time as a function of particle concentration in cell culture media for both NP-dextran and NP-PEG-methotrexate conjugates. The NP-PEG-methotrexate conjugates have a much shorter T₂ relaxation time (high relaxivity) than the dextran-nanoparticles due to enhanced magnetism which resulted from greater uptake of NP-PEG-methotrexate conjugates by the 9L glioma cells. These results further confirm the targeting role of the NP-PEG-methotrexate for 9L cells and its detectability by MRI.

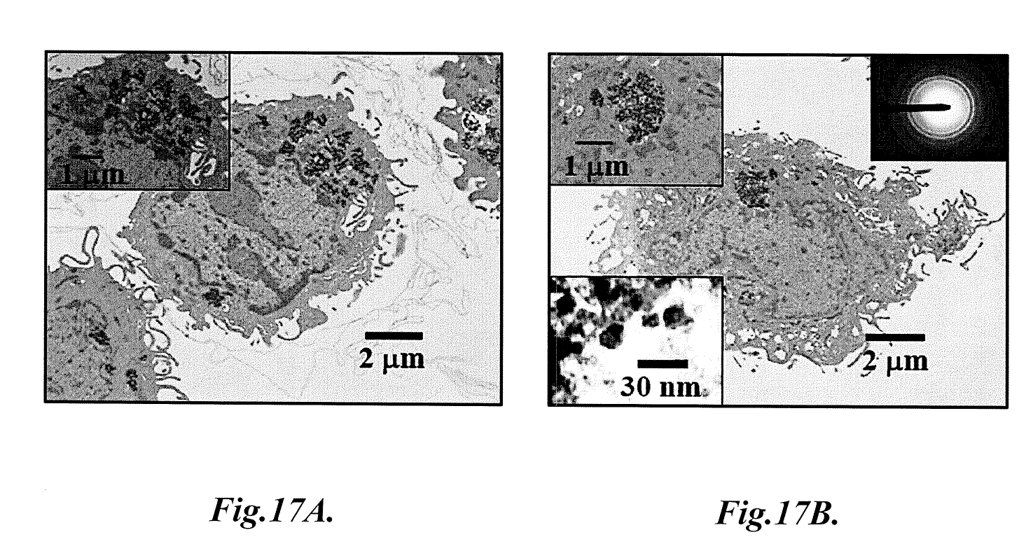

Uptake of NP-PEG-methotrexate conjugates by 9L cells were visualized by TEM imaging. TEM imaging also revealed the location of the nanoparticle conjugates inside the cells. For TEM imaging, cells were first cultured with NP-PEG-methotrexate at a concentration of 0.1 mg/mL for 24 hours, and then rescued with 10 μg/mL Leucovorin. FIG. 17 shows TEM images of (A) 9L cells incubated with NP-PEG-methotrexate for 24 hours, and (B) 9L cells incubated with NP-PEG-methotrexate for 24 hours and then exposed to Leucovorin (LV) for 144 hours. The NP-PEG-methotrexate conjugates are seen in the cellular cytoplasm in both cases. The cell rescued with LV appeared to contain a much lower concentration of NP-PEG-methotrexate in its cytoplasm than the cell without undergoing the rescue. This reduction in iron concentration is likely due to the continued cellular proliferation after LV rescue, which reduced the average particle number per cell as a result of the increased cell number.

The electron diffraction pattern shown as an inset of FIG. 17B indicates that the nanoparticle conjugates in the cellular cytoplasm did not loose their magnetite crystal structure following 144 hours in culture. The clinical implication of this long-term retention of the nanoparticle conjugate inside target cells and its magnetite crystal structure is that the NP-PEG-methotrexate conjugate may serve as a contrast enhancement agent for a prolonged time after its internalization, which may be potentially applied for sequential prognosis, preoperation diagnostics, intraoperative monitoring, and post-operation analysis with MRI. A higher magnification image (an inset of FIG. 17B) reveals that the individual particle conjugates within the lysosomes have a size of approximately 10-15 nanometers.

The methotrexate-modified nanoparticle systems in the invention has a number of combined advantages in view of its therapeutic functionality to treat tumors:

(1) the methotrexate delivery system enables real-time monitoring of drug delivery to the target tumor through MRI, thus allowing physicians to access the efficacy of their treatment utilizing MRI. The invention may be a viable solution to chemotherapeutic drug delivery for treatment of cancers that highly express folate receptors, and yet serves as a contrast agent for MRI. The methotrexate-modified nanoparticle demonstrated an increased MRI contrast enhancement through intracellular uptake by target cells.

(2) by covalently modifying the surface of the nanoparticle via a pH sensitive bond, such as a peptide bond, methotrexate is not released from the surface of the nanoparticles under intravenous conditions. Instead, cleavage of the amide bond occurs under conditions present in the lysosomal compartment, namely, at low pH and in the presence of lysozymes, a typical environment inside the target cells. Due to the overexpressed folate receptor on target cells as opposed to healthy cells, this release mechanism will greatly reduce toxic effects of methotrexate to healthy tissues within the body. The increased uptake of the methotrexate conjugated nanoparticles in tumor cells over-expressing the folate receptor has been demonstrated.

(3) Use of PEG as a linker between nanoparticles and the targeting agent, methotrexate, may prevent nanoparticle aggregation, potentially improve particle circulation time in the blood, reduce uptake of nanoparticles by the mononuclear phagocyte system, and therefore, enable sufficient amount of the methotrexate-modified nanoparticles to be specifically delivered to tumor cells, detectable by MRI, and induce cellular apoptosis.

The following examples are provided for the purpose of illustrating, not limiting, the invention.

### EXAMPLES

### Example 1

### Synthesis of Methotrexate-Modified Nanoparticles with Alkyl Linker

Surface modification of nanoparticles with 3-aminopropyl trimethoxysilane and methotrexate. Magnetite nanoparticles were synthesized by a co-precipitation method with minor modifications outlined previously (Chang, Y.; Kohler, N.; Zhang, M., Surface Modification of Superparamagnetic Magnetite Nanoparticles and Their Intracellular Uptake, Biomaterials 2002, 23, 1553-1561). The magnetite nanoparticles were surface-modified with methotrexate via a chemical scheme outlined in FIG. 1. The nanoparticles were first surface-modified with 3-aminopropyltrimethoxysilane (APS) to form a self-assembled monolayer (SAM) and subsequently conjugated with methotrexate through amidation between the carboxylic acid end group on methotrexate and the amine groups on the particle surface. The methotrexate conjugation reaction may occur through either the a or B carboxylic acid groups on the glutamic acid residue. One milliliter of APS was added to a colloidal suspension of 200 mg of magnetite nanoparticles in 100 mL of toluene dried using molecular sieves. The nanoparticles were sonicated in a sonicating bath for 4 hours at 60° C. The resulting aminated nanoparticles were then isolated using a rare earth magnet and washed twice with 200 proof ethanol and twice with deionized water. To conjugate the nanoparticles with methotrexate, free methotrexate was dissolved in 17 mL of DMSO (10 mM) due to the limited solubility of methotrexate in water. The solution of methotrexate was then mixed with a 17 mL aqueous solution of 1-ethyl-3- (3-dimethylamino-propyl) carbodiimide (EDC) (75 mM) and N-hydroxysuccinimide (NHS) (15 mM). The pH of the solution was adjusted to 8.2 by addition of 1.0 M NaOH. The resulting suspension was agitated overnight at 37° C. in the dark. Following methotrexate conjugation, the modified particles, NP-propyl-methotrexate, were again isolated with a rare earth magnet and washed 5 times with deionized (DI) water.

Characterization of methotrexate-modified nanoparticles with FTIR. Fourier transform infrared (FTIR) spectra were acquired using a Nicolet 5-DXB FTIR spectrometer with a resolution of 4 cm⁻¹. To characterize the amine SAM and methotrexate on the nanoparticle surface, 2 mg of dried nanoparticles was added to 200 mg of KBr. The mixture was pressed into a pellet for analysis.

### Example 2

### Synthesis of Methotrexate-Modified Nanoparticles with PEG Linker

Dulbecco's phosphate buffered saline (PBS), N-Hydroxysuccinimide 97% (NHS), 1-Ethyl-3-(3-dimethylamino-propyl) carbodiimide (EDAC), iron (II) and iron (III) chloride were purchased from Sigma (St. Louis, Mo.). All other solvents were purchased from Fisher Scientific (Hampton, N.H.) or Aldrich (Milwaukee, Wis.).

Magnetite nanoparticles were synthesized by a co-precipitation method. The acidic iron chloride solution was prepared by dissolution of 3.09 g (24.37 mM) of FeCl₃ and 5.2 g (32.06 mM) of FeCl₂ in 100 mL of 0.96 M hydrochloric acid solution. The resultant solution was placed in a sonicating bath and stirred for five hours during which time 500 mL of 1.5M NaOH solution was introduced drop-wise via a peristaltic pump. Particle synthesis temperature was controlled by an external water-circulator connected to the sonicating bath containing a solution of 50% ethylene glycol and 50% DI water. To prevent nanoparticles from oxidation following nucleation, nitrogen gas was bubbled into the solution to reduce the oxygen partial pressure. Following the precipitation, the nanoparticles were isolated with a 1″ rare earth magnet, washed three times with deionized water, and resuspended using a Fisher Model 500 ultrasonic dismembrator with a ½″ tip operating at 90% power for 5 minutes. The particles were then magnetically isolated by placing the particles on a rare earth magnet for 2 hours. To ensure the particle stability, the particles were washed and sonicated in 2 M HNO₃ in deionized water. The particles were again isolated with a rare earth magnet and washed twice with deionized water. The pH was adjusted to 5 by addition of 1 M NaOH. NP-Dextran conjugates (as a control) were synthesized following the published protocol.

Immobilization of TFEE-PEG silane. To immobilize the TFEE-PEG silane on the nanoparticle surface, the solvent was exchanged to toluene following the nanoparticle precipitation. 100 mg of native nanoparticles were isolated on a rare earth magnet. The particles were then washed twice with 100 mL of absolute ethanol and sonicated in a sonicating bath. After sonication, the particles were again isolated using a rare earth magnet, washed twice with toluene, and dried over molecular sieves. The particles were then sonicated for two minutes using a Fisher Model 500 ultrasonic dismembrator operating at 60% power with a ⅛″ microtip. One mL of the TFEE-PEG silane was then added to the suspension. The particles were sonicated for 12 hours at room temperature and 4 hours at 60° C. in an ultrasonic bath. After sonication, the particles were isolated on a rare earth magnet and washed twice with toluene and resuspended in 100 mL of toluene. Following the immobilization of the TFEE-PEG silane on the nanoparticle surface, ethylene diamine was used to convert the carboxylic acid group to a primary amine. One mL of ethylene diamine was added to 100 mL of nanoparticle suspension in toluene and sonicated for 4 hours at room temperature. Following the reaction, the particles were isolated and washed twice with 100 mL of absolute ethanol and twice with deionized water.

Immobilization of methotrexate to PEG chain terminus. The primary amine on the PEG chain terminus allows the subsequent immobilization of methotrexate through an amide bond. To immobilize the methotrexate, 33 mg of methotrexate, 22 mg of N-Hydroxysuccinimide, and 159 mg of EDC were dissolved in 17 mL of DMSO and 20 mL of deionized water. The pH was adjusted to 6 and the mixture was allowed to react for 30 minutes. To produce a succinimidyl ester on the methotrexate following the reaction, the solution was mixed with 15 mg of amine-terminal PEG silane nanoparticles. The particles were sonicated for 2 minutes, the pH was adjusted to 8, and the particles were incubated overnight at 37° C. on an orbital shaker. Following the immobilization of methotrexate, the particles were washed 5 times with 100 mL of deionized water and 3 times with 20 mM sodium citrate buffer at pH 8, and suspended in citrate buffer for use in the experiments.

Fourier transform infrared spectroscopy (FTIR) of methotrexate immobilized nanoparticles. FTIR spectra were acquired using a Nicolet 5-DXB FTIR spectrometer at a resolution of 4 cm⁻¹. To characterize the amine SAM and methotrexate on the nanoparticle surface, 2 mg of dried powder was added to 200 mg of KBr. The mixture was pressed into a pellet for analysis.

### Example 3

### Analysis of Methotrexate Release from Magnetite Nanoparticles

To simulate intracellular lysosomal conditions, methotrexate modified nanoparticles, NP-propyl-methotrexate, at a concentration of 0.1 mg/mL were suspended in a solution of 0.1 mg/mL crude protease from bovine pancreas (Sigma) in 5 mL of phosphate-buffered saline (PBS) solution at 37° C. under constant stirring. The solution pH was adjusted by the titration of 1.0M HCl and 1.0 M NaOH to achieve pH's of 2, 3, 4, 5.6, and 7.44 respectively. Following incubation for 8, 24, 48, and 72 hours, the nanoparticle suspensions were centrifuged at 2000 rpm to isolate the particles from methotrexate, PBS, and protease solutions. Methotrexate cleavage from nanoparticles was then quantified with UV spectroscopy at wavelength of 304 nm.

### Example 4

### Drug Efficacy of Methotrexate-Modified Nanoparticles in Hela and MCF-7 Cells

Human breast cancer cells (MCF-7), and human cervical cancer cells (HeLa) were obtained from the Fred Hutchinson Cancer Research Center, Seattle, Wash. Cells were grown in T-75 flasks with RPMI Media (Invitrogen) supplemented with 10% fetal calf serum, 5958 mg/mL HEPES, 300 mg/mL L-Glutamine, 50 ug/mL streptomycin and 50 IU/mL penicillin. Cells were cultured at 37° C. in a humidified atmosphere with 5% CO₂ with the medium being changed every third day. The cells were transferred to RPMI-1640 folate free medium (Invitrogen) 24 hours prior to plating. The cells were then subcultured in 24-well plates at a concentration of 25,000 cells/mL in RPMI-1640 folate-free medium. NP-propyl-methotrexate nanoparticles were mixed in RPMI-1640 folate-free media at iron concentrations of 0.01, 0.025, 0.05, 0.075, and 0.1 mg/mL. In addition, a control media containing soluble methotrexate was prepared at a concentration of 2 μg/mL. Cells were also cultured in 1 mL medium without nanoparticles as the control. The cell culture proceeded for 24, 48, 72, 96, and 120 hours respectively at which time, the cells were washed three times with 1 mL of Hank's Balanced Salt Solution (HBSS, Invitrogen), detached with 500 μL of 0.25% trypsin-EDTA (Sigma), and resuspended in 1 mL of Phosphate Buffered Saline (PBS) supplemented with 10% FBS. Cell viability was determined by cell count via a model ZI Beckman particle counter.

### Example 5

### Transmission Electron Microscopy (TEM) of Cells Exposed to Methotrexate Nanoparticles

Cells were cultured at 37° C. in a humidified atmosphere with 5% CO₂. The medium was changed every third day. When the cells achieved confluence, they were incubated with 10 mL of folate-free cellular medium (Invitrogen, Corp) containing 300 mg/mL L-Glutamine, 10% fetal calf serum, 50 IU/mL penicillin and 50 μg/mL streptomycin. Following 24 hours of incubation, NP-propyl-methotrexate nanoparticles were introduced into the culture medium at an iron concentration of 0.1 mg/mL. Control cells were cultured in folate-free medium without nanoparticles. After 1 day in culture with nanoparticles, the cells were washed once with 5 mL of Versene (Invitrogen) and twice with HBSS, followed by detachment with trypsin. The cells were then centrifuged and resuspended in 5 mL of Karnovsky's Fixative for 24 hours. Following fixation, the cells were processed in agar and embedded in epoxy for sectioning. Cell sections were stained with osmium tetroxide, lead citrate, and uranyl acetate for transmission electron microscopy (TEM) contrast enhancement. Cell and nanoparticle images were taken using a Phillips 420 TEM microscope at 100 kV.

### Example 6

### Intracellular Uptake of Magnetite Nanoparticles

To quantify the cellular uptake of methotrexate nanoparticles by cancer cells, HeLa and MCF-7 cells were grown in T-75 flasks and rat cardiomyocyte cells (Cell Applications, Inc.) on 12-well plates. HeLa and MCF-7 cells were cleaved using 0.25% trypsin-EDTA solution in HBSS and were seeded in 12-well tissue culture plates at a concentration of 106 cells/mL. Prior to the uptake experiments, all cells were cultured in RPMI-1640 folate-free medium in the 12-well plates for 24h. The NP-propyl-methotrexate nanoparticle stock solution in DI water was sonicated for 4 minutes and dispersed into RPMI-1640 folate-free medium at a concentration of 0.1 mg/mL. The cells were then grown with 2 mL of the methotrexate-nanoparticle medium for 2 hours for particle internalization. Following culture with the nanoparticles, the cells were washed once with 1.0 mL of RPMI-1640 folate-free medium, twice with 1.0 mL of PBS supplemented with 10% FBS. A 100 μL aliquot of the cell suspension in PBS was then dispersed in 9.9 mL of Isoton solution, and cells were counted using a Beckman ZI particle counter. To lyse the cells, 100 μL of concentrated HCl was added to the 900 μL of remaining cell suspension and incubated for 1 hour at 70° C. The resulting intracellular iron concentration was determined by inductively coupled plasmon resonance spectroscopy (ICP).

### Example 7

### Quantification of Methotrexate Immobilized on the Nanoparticle

Ultra violet (UV) absorbance spectroscopy was used to determine the amount of methotrexate immobilized on the nanoparticle surface. A stock 10 mM solution of methotrexate in dimethyl sulfoxide (DMSO) was used as a standard. The methotrexate was serially diluted to concentrations of one to five μg/mL in DI water. The methotrexate absorbance was measured at 304 nm, and a standard least squares regression curve was utilized to create a linear fit of the data.

2.3 mg/mL methotrexate immobilized nanoparticles, NP-PEG-methotrexate, were dispersed in 1 mL of phosphate buffered saline. 8.25 ng of proteinase K enzyme was dispersed in the solution and incubated at 37° C. in a water bath. The nanoparticle suspension was centrifuged at 15,000 RPM to pelletize the nanoparticles from solution. The supernatant was measured at 304 nm and compared to the standard curve created previously.

### Example 8

### Cell Viability of Glioma Cells Exposed to Free Methotrexate

Rat glioma cells (9L) were grown in T-75 flasks with RPMI Media (Invitrogen) supplemented with 10% fetal calf serum, 5958 mg/mL HEPES, 300 mg/mL L-Glutamine, 50 μg/mL streptomycin and 50 IU/mL penicillin. The cells were transferred to and subcultured in 24 well plates at a concentration of 10000 cells/mL in RPMI-1640 folate free medium and allowed to attach overnight. A stock solution of methotrexate was prepared in DMSO at a concentration of 10 mM and diluted into RPMI folate free medium at concentrations of 0.05, 0.1, 0.5, 1, 5 and 10 μg/mL. The cells were cultured for 24, 48, 72, 96, and 120 hours respectively at which times the cells were washed twice with phosphate buffered saline (PBS), detached with 0.25% Trypsin EDTA (Sigma), and resuspended in 1 mL of (PBS) supplemented with 10% FBS to prevent cell lysing. Cell viability was determined by cell count with a ZI Beckman particle counter.

### Example 9

### Cytotoxicity of Magnetite Nanoparticles to 9L Cells

To determine the cytotoxicity of NP-PEG-methotrexate conjugates, 9L cells were plated into 24 well plates at a concentration of 10,000 cells/well. Following 24 hours in culture, the cells were washed twice with PBS and incubated with the NP-PEG-methotrexate conjugates at concentrations of 0.001 mg/mL, 0.01 mg/mL, and 0.1 mg/mL iron. Cells were washed three times with 1.0 mL of Hank's balanced salt solution (HBSS, Invitrogen), detached with 500 μL of 0.25% trypsin-EDTA (Sigma), and resuspended in 1.0 mL of PBS supplemented with 10% FBS. Cell viability was determined by cell count with a ZI Beckman particle counter.

### Example 10

### Leucovorin Rescue of Apoptotic Cells Induced by Methotrexate Nanoparticles

To identify the cause of cytotoxicity, 9L cells were plated in 24 well plates at a concentration of 7000 cells/well and allowed to attach for 24 hours. The NP-PEG-methotrexate nanoparticles were then dispersed in RPMI folate free medium at a concentration of 0.1 mg/mL and incubated with the cells for 24 hours. Free methotrexate at a concentration of 0.22 μg/mL was also incubated with the cells and used as a control. Following a 24 hour exposure to either methotrexate or methotrexate nanoparticles, the cells were washed twice with PBS and twice with Hank's Balanced Salt Solution (HBSS), and incubated with 10 μg/mL Leucovorin in folate free medium for 24, 48, 72, 110 and 144 hours respectively and counted at each time interval.

### Example 11

### Uptake of Nanoparticle Conjugates by Tumor Cells

To measure the uptake of NP-PEG-methotrexate conjugates by target cells, 9L cells were plated in T-25 flasks at a concentration of 500,000 cells/well and allowed to grow to confluency. 24 hours prior to the uptake experiment, the cells were washed twice with PBS and incubated in RPMI folate free medium. The nanoparticle conjugates were dispersed into RPMI folate free medium and the cells were incubated with the nanoparticle conjugates at 37°, 5% CO₂ for 2 hours. After the uptake, the cells were washed once with Versene (Invitrogen), twice with PBS, and three times with HBSS. The cells were then cleaved with 0.25% Trypsin EDTA (Invitrogen) and dispersed into 2 mL of 10% FBS in PBS. 100 μL of the cell suspension was counted using a Beckman ZI coulter counter. 100 μL of concentrated HCl was added to 1.5 mL of the remaining cell suspension and incubated at 80° C. for 2 hours to lyse the cells and digest the nanoparticles. The uptake of nanoparticle conjugates was measured with a Jarrell Ash Inductively Coupled Plasmon Resonance Optical Emission Spectrometer (ICP) using a 10 ppm standard.

### Example 12

### Magnetic Resonance Imaging (MRI)

Samples for phantom imaging were prepared by suspending 10⁶ cells in 50 μl of 1% low-melting agarose (BioRad, Hercules, Calif.). Cell suspensions were loaded into a pre-fabricated 12-well agarose sample holder and allowed to solidify at 4° C. MR images were acquired using a 4.7-Tesla Varian MR spectrometer (Varian, Inc., Palo Alto, Calif.) and a Bruker magnet (Bruker Medical Systems, Karlsruhe, Germany) equipped with a 5 cm volume coil. A spin-echo multisection pulse sequence was selected. Repetition time (TR) of 3000 msec and variable echo times (TE) of 15-90 msec were used. The spatial resolution parameters were set as follows: an acquisition matrix of 256×128, field of view of 4×4 cm, section thickness of 1 mm, and 2 averages. Regions of interest (ROI) of 5.0 mm in diameter were placed in the center of each sample image to obtain signal intensity measurements using NIH ImageJ. T₂ values were obtained using a built-in Varian macro, “t2” fit program, to generate a T₂ map of the acquired images.

### Example 13

### Cell Images by Transmission Electron Microscopy (TEM)

The cells were centrifuged and resuspended in 5 mL of Kamovsky's fixative for 24 hours. Following fixation, the cells were processed in agar and embedded in epoxy for sectioning. Cell sections were stained with osmium tetroxide, lead citrate, and uranyl acetate for TEM contrast enhancement. Cell and nanoparticle images were taken using a Phillips 420 TEM microscope operated at 100 kV.

1. A methotrexate-modified nanoparticle, comprising:
(a) a core having a surface, the core comprising a material having magnetic resonance imaging activity;
(b) a methotrexate; and
(c) a linker covalently coupling the methotrexate to the surface.

2. The particle of claim 1, wherein the material having magnetic resonance imaging activity comprises a metal oxide selected from the group consisting of ferrous oxide, ferric oxide, silicon oxide, polycrystalline silicon oxide, aluminum oxide, germanium oxide, zinc selenide, tin dioxide, titanium dioxide, indium tin oxide, gadolinium oxide, and mixtures thereof.

3. The particle of claim 1, wherein the core comprises a material selected from the group consisting of silicon nitride, stainless steel, titanium, nickel titanium, and mixtures thereof.

4. The particle of claim 1, wherein the methotrexate-modified particle has from about 20 to about 500 methotrexates/particle.

5. The particle of claim 1, wherein the linker comprises an alkyl moiety.

6. The particle of claim 1, where the linker comprises a propyl moiety.

7. The particle of claim 1, wherein the linker comprises a poly(ethylene glycol) moiety.

8. The particle of claim 1, wherein the linker comprises a 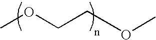
moiety, wherein n is an integer from about 10 to about 1000.

9. The particle of claim 8, wherein n is 10.

10. The particle of claim 1, wherein the methotrexate is linked to the surface through a pH sensitive covalent bond.

11. The particle of claim 1, wherein the methotrexate is linked to the surface through a peptide bond.

12. The particle of claim 1, wherein the core has a diameter of from about 5 nm to about 20 nm.

13. The particle of claim 1, wherein modified particle has a diameter of from about 50 nm to about 200 nm.

14. A pharmaceutical composition, comprising a methotrexate-modified particle of claim 1 and a pharmaceutically acceptable carrier.

15. A method for differentiating neoplastic tissue from non-neoplastic tissue, comprising:
(a) contacting a tissue of interest with a methotrexate-modified nanoparticle having affinity and specificity for tumor cells over-expressing folate receptor; and
(b) measuring the level of binding of the methotrexate-modified nanoparticle, wherein an elevated level of binding, relative to normal tissue, is indicative that the tissue is neoplastic.

16. The method of claim 15, wherein measuring the level of binding of the methotrexate-modified nanoparticle comprises magnetic resonance imaging.

17. A method for detecting a tissue over-expressing folate receptor, comprising:
(a) contacting a tissue of interest with a methotrexate-modified nanoparticle having affinity and specificity for folate receptor; and
(b) measuring the level of binding of the methotrexate-modified nanoparticle, wherein an elevated level of binding, relative to normal tissue, is indicative of the presence of a tumor over expressing folate receptor.

18. The method of claim 17, wherein measuring the level of binding of the methotrexate-modified nanoparticle comprises magnetic resonance imaging.

19. A method for treating a tumor in a patient, comprising administering to a patient in need thereof an effective amount of a pharmaceutical composition comprising a methotrexate-modified nanoparticle of claim 1 and a pharmaceutically acceptable carrier.

20. A method for inhibiting invasive activity of neoplastic cells, comprising administering to neoplastic cells an effective amount of a pharmaceutical composition comprising a methotrexate-modified nanoparticle of claim 1 and a pharmaceutically acceptable carrier.

21. A method for treating a tumor in a patient, comprising:
(a) administering a pharmaceutical composition to a patient, wherein the pharmaceutical composition comprises a pharmaceutically acceptable carrier and an amount of a methotrexate-modified nanoparticle sufficient for treatment;
(b) monitoring the amount of methotrexate-modified nanoparticles delivered to the target tumor by magnetic resonance imaging.
(c) accessing the efficacy of the treatment by analyzing the data from the magnetic resonance imaging.
